(19) United States
(12) Patent Application Publication
(10) Pub. No.: US 2008/0312295 A1
(43) Pub. Date: Dec. 18, 2008

(54) Pesticidal Mixtures

(75) Inventors: Ronald Wilhelm, Hofheim (DE); Jurgen Langewald, Mannheim (DE); Douglas D. Anspaugh, Apex, NC (US)

Correspondence Address: BRINKS, HOFER, GILSON & LIONE, 2801 SLATER ROAD, SUITE 120, MORRISVILLE, NC, 27560, US

(73) Assignee: BASF SE, Ludwigshafen (DE)

(21) Appl. No.: 12/063,330

(22) PCT Filed: Aug. 8, 2006
(86) PCT No.: PCT/EP2006/065135

§ 371 Date: Feb. 8, 2008

Related U.S. Application Data
(60) Provisional application No. 60/707,312, filed on Aug. 11, 2005.
(60) Provisional application No. 60/833,459, filed on Jul. 26, 2006.

(51) Int. Cl. A01N 43/40; A61K 31/44; A01P 5/00; A01P 7/00

(52) U.S. Cl. 514/355

(57) ABSTRACT

Pesticidal mixtures comprising, as active components,
A) a phenylsemicarbazone compound of the formula (I),

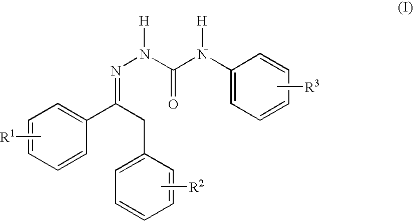

where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II),

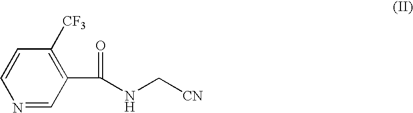

or an agriculturally acceptable salt thereof.

The invention relates to mixtures comprising pesticidal phenylsemicarbazones and the use of such mixtures for controlling pests.

One typical problem arising in the field of pest control lies in the need to reduce the dosage rates of the active ingredient in order to reduce or avoid unfavorable environmental or toxicological effects whilst still allowing effective pest control.

Another problem encountered concerns the need to have available pest control agents which are effective against a broad spectrum of pests.

There also exists the need for pest control agents that combine knock-down activity with prolonged control, that is, fast action with long lasting action.

Another difficulty in relation to the use of pesticides is that the repeated and exclusive application of an individual pesticidal compound leads in many cases to a rapid selection of pests which have developed natural or adapted resistance against the active compound in question. Therefore there is a need for pest control agents that help pre-vent or overcome resistance.

It is therefore an object of the invention to provide pesticidal mixtures which solve the problems of reducing the dosage rate and/or enhancing the spectrum of activity and/or combining know-down activity with prolonged control and/or to resistance management.

EP-A 0 462 456 discloses phenylcarbazones having a wide insecticidal spectrum. However, these compounds do not always show a completely satisfactory performance with respect to the above mentioned problems.

It has now been found that by mixing phenylsemicarbazones with flonicamid the object of the invention can be achieved at least in certain aspects.

Accordingly, in one aspect of the invention there are provided pesticidal mixtures comprising
A) a phenylsemicarbazone compound of the formula (I),

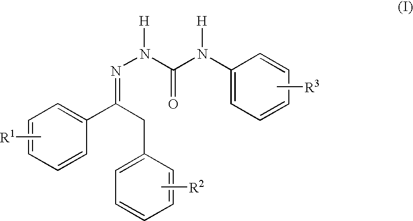

where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II),

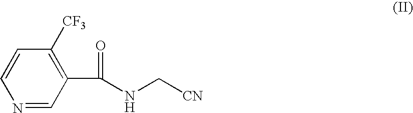

or an agriculturally acceptable salt thereof.

The common name of the compound of formula (II) is flonicamid (N-cyanomethyl-4-(trifluoromethyl)nicotinamide).

This invention also relates to a method for protecting plants from attack or infestation by pests, namely insects, arachnids or nematodes, using mixtures of the compound (I) with the compound (II) (flonicamid), to a method for controlling pests, namely harmful arthropods, like insects and arachnids, or nematodes using mixtures of the compound (I) with flonicamid, and to the use of the compound (I) and flonicamid for preparing such mixtures, and compositions comprising these mixtures.

In the context of the invention, the term plant refers to an entire plant, a part of the plant or the propagation material of the plant, especially the seed.

Besides, the invention also relates to a method for treating, controlling, preventing or protecting a warm-blooded animal or a fish against infestation or infection by pests using the inventive mixtures.

The 1-phenylsemicarbazones of formula (I), their preparation and their action against arthropods are known (e.g. EP-A 0 482 456).

Flonicamid, its preparation and its action against pests is likewise known from the literature (EP-A 0 580 374).

Mixtures, active against pests, of flonicamid or its derivatives and various active compounds are described in a general manner in EP-A 0 580 374. The favourable synergistic effect of these mixtures is not mentioned in this document.

Preferred compounds of formula (I) are those, where

R¹ is C₁-C₄-haloalkyl, more preferred C₁-C₄ fluoroalkyl, in particular CF₃;

R² is CN; and

R³ is C₁-C₄-haloalkoxy, more preferred C₁-C₄-fluoroalkoxy, in particular OCF₃.

“Halo” means F, Cl, Br and I.

Particularly preferred is the compound of formula (I), where R¹ is 3-CF₃, R² is 4-CN and R³ is 4-OCF₃, (Ia),

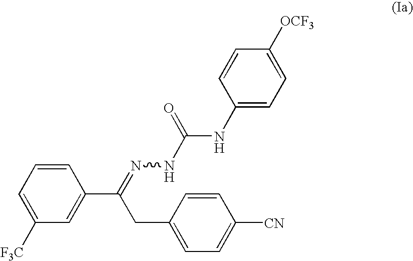

which has the common name metaflumizone. Metaflumizone and its preparation is described, e.g., in EP-A 462 456.

“Agriculturally acceptable salts” of the compounds (I) or (II) can be formed in a customary manner, e.g. by reaction with an acid of the anion in question and include adducts of compounds (I) or (II) with maleic acid, dimaleic acid, fumaric acid, difumaric acid, methane sulfenic acid, methane sulfonic acid, and succinic acid. Moreover, included are those salts that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide.

Preferably, the mixture of the invention is a mixture of metaflumizone and flonicamid.

Preferably, the mixture of the invention comprises components (A) and (B) in synergistically effective amounts.

Preferably, the mixture of the invention comprises components (A) and (B) in a synergistically effective ratio.

When preparing the mixtures, it is preferred to employ the pure active compounds (I) and (II), to which further active compounds, also against harmful fungi or else herbicidal or growth-regulating active compounds or fertilizers can be added.

The mixtures of compounds (I) and (II), or the compounds (I) and (II) used simultaneously, that is jointly or separately, exhibit outstanding action against pests from the following orders:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles mini-mus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa

thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae sp such as Aculus schlechtendali, Phyllocoptrata oleivora and Eriophyes sheldoni; Tarsonemidae sp such as Phytonemus pallidus and Polyphagotarsonemus latus; Tenuipalpidae sp such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa, ticks (Ixodida), e.g. Phipicephalus sanguineus, or mites, such as Mesostigmata, e.g. Ornithonyssus bacoti and Dermanyssus gallinae, Prostigmata, e.g. Pymotes tritici, or Astigmata, e.g. Acarus siro,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

Plant parasitic nematodes such as root-knot nematodes, Meloidogyne arenaria, Meloidogyne chitwoodi, Meloidogyne exigua, Meloidogyne hapla, Meloidogyne incognita, Meloidogyne javanica and other Meloidogyne species; cyst nematodes, Globodera rostochiensis, Globodera pallida, Globodera tabacum and other Globodera species, Heterodera avenae, Heterodera glycines, Heterodera schachtii, Heterodera trifolii, and other Heterodera species; seed gall nematodes, Anguina funesta, Anguina tritici and other Anguina species; stem and foliar nematodes, Aphelenchoides besseyi, Aphelenchoides fragariae, Aphelenchoides ritzemabosi and other Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicotylenchus dihystera, Helicotylenchus multicinctus and other Helicotylenchus species, Rotylenchus robustus and other Rotylenchus species; sheath nematodes, Hemicycliophora species and Hemicriconemoides species; Hirshmanniella species; lance nematodes, Hoplolaimus columbus, Hoplolaimus galeatus and other Hoplolaimus species; false root-knot nematodes, Nacobbus aberrans and other Nacobbus species; needle nematodes, Longidorus elongates and other Longidorus species; pin nematodes, Paratylenchus species; lesion nematodes, Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, Radopholus similis and other Radopholus species; reniform nematodes, Rotylenchulus reniformis and other Rotylenchulus species; Scutellonema species; stubby root nematodes, Trichodorus primitivus and other Trichodorus species; Paratrichodorus minor and other Paratrichodorus species; stunt nematodes, Tylenchorhynchus claytoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species; and other plant parasitic nematode species.

The mixtures according to the invention are especially useful for the control of pests of the orders Coleoptera, Diptera, Hemiptera, Acarina, Lepidoptera, Thysanoptera, Homoptera, Isoptera and Orthoptera, specifically for the control of those pests from these orders mentioned in the list above.

They are particularly useful for the control of pests from the mentioned orders which are disclosed in the experimental section below.

They are also useful for preparing compositions for the control of the said pests. The mixtures according to the invention or the compounds (I) and (II) can be in the form of pesticidal compositions, further comprising a liquid or solid carrier, such as customary formulations, for example solutions, emulsions, suspensions, dusts, powders, pastes and granules. The application form depends on the particular purpose; in each case, it should ensure a fine and uniform distribution of the compounds (I) and (II).

The formulations are prepared in a known manner, for example by extending the active compounds with customary formulation aids, such as solvents and/or carriers, if desired using emulsifiers and dispersants and further customary additives. Solvents/auxiliaries which are suitable include:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the mixture of the active compounds. The mixture of the active compounds are employed in a purity of from 90% to 100%, preferably 95% to 100% (according to NMR spectrum).

The following are examples of formulations: 1. Products for dilution with water

### A) Soluble Concentrates (SL, LS)

10 parts by weight of the active compounds are dissolved in water or in a water-soluble solvent. As an alternative, wetters or other auxiliaries are added. The active compounds dissolve upon dilution with water.

### B) Dispersible Concentrates (DC)

20 parts by weight of the active compounds are dissolved in cyclohexanone with addition of a dispersant, for example polyvinylpyrrolidone. Dilution with water gives a dispersion.

### C) Emulsifiable Concentrates (EC)

15 parts by weight of the active compounds are dissolved in xylene with addition of calcium dodecylbenzenesulfonate and castor oil ethoxylate (in each case 5% strength). Dilution with water gives an emulsion

### D) Emulsions (EW, EO, ES)

40 parts by weight of the active compounds are dissolved in xylene with addition of calcium dodecylbenzenesulfonate and castor oil ethoxylate (in each case 5% strength). This mixture is introduced into water by means of an emulsifier (Ultraturax) and made into a homogeneous emulsion. Dilution with water gives an emulsion.

### E) Suspensions (SC, OD, FS)

In an agitated ball mill, 20 parts by weight of the active compounds are comminuted with addition of dispersant, wetters and water or an organic solvent to give a fine active compound suspension. Dilution with water gives a stable suspension of the active compounds.

### F) Water-Dispersible Granules and Water-Soluble Granules (WG, SG)

50 parts by weight of the active compounds are ground finely with addition of dispersants and wetters and made into water-dispersible or water-soluble granules by means of technical appliances (for example extrusion, spray tower, fluidized bed). Dilution with water gives a stable dispersion or solution of the active compounds.

### G) Water-Dispersible Powders and Water-Soluble Powders (WP, SP, WS)

75 parts by weight of the active compounds are ground in a rotor-stator mill with addition of dispersant, wetters and silica gel. Dilution with water gives a stable dispersion or solution with the active compound(s).

### 2. Products to be Applied Undiluted

### H) Dustable Powders (DP, DS)

5 parts by weight of the active compounds are ground finely and mixed intimately with 95% of finely divided kaolin. This gives a dustable product. Ps I) Granules (GR, FG, GG, MG)

0.5 part by weight of the active compounds are ground finely and associated with 95.5% carriers. Current methods are extrusion, spray-drying or the fluidized bed. This gives granules to be applied undiluted.

### J) ULV Solutions (UL)

10 parts by weight of the active compounds are dissolved in an organic solvent, for example xylene. This gives a product to be applied undiluted.

In a preferred embodiment of the invention there is provided an emulsifiable concentrate (EC) formulation, comprising

a) the mixture according to the invention;
b) a solvent system, comprising
b1) γ-butyrolactone,
b2) one or more aliphatic and/or aromatic ketone, and
b3) optionally one or more aromatic hydrocarbon;
c) one or more emulsifier;
d) optionally, further formulation additives.

The preferred EC formulation generally comprises 0.1 to 30% by weight, preferably 8 to 18% by weight, in particular 10 to 15% by weight, of the compound of formula (I).

The preferred EC formulation generally comprises 6 to 97% by weight, preferably 10 to 90% by weight, in particular 25 to 80% by weight, of the solvent system (b).

γ-Butyrolactone, component (b1) of the solvent system is a commercially available solvent which can be obtained, e.g., from BASF Aktiengesellschaft, Germany.

γ-Butyrolactone is generally contained in an amount of 2 to 90% by weight, preferably 10 to 75% by weight, in particular 20 to 40% by weight of the formulation.

Suitable ketones as component (b2) of the solvent system include C₁ to C₂₀ aliphatic, cycloaliphatic and aromatic ketones.

Preferred are C₅ to C₁₈ alkanones, in particular 2-heptanone, mesityl oxide, cyclohexanone, isophorone, frenchone and acetophenone.

In a preferred embodiment component (b2) comprises two ketones, preferably acetophenone and an C₅-C₁₈ alkanone, in particular acetophenone and 2-heptanone.

Ketone component (b2) generally amounts to from 4 to 92% by weight, preferably 15 to 80% by weight of the formulation.

In the preferred embodiment acetophenone generally amounts to from 2 to 70% by weight, preferably 5 to 40% by weight, in particular 20 to 30% by weight of the formulation.

The aliphatic ketone, preferably 2-heptanone, generally amounts to from 2 to 90% by weight, preferably 10 to 40% by weight, in particular 10 to 30% by weight of the formulation.

All listed ketones are commercially available products.

Optionally, the solvent system comprises aromatic hydrocarbons as component (b3). Preferably, mixtures of alkylaromatics, in particular alkylbenzenes and alkylnaphthalenes, whose alkyl groups have 1 to 20 carbon atoms, are employed. Such mixtures are commercially available, e.g. as the Solvesso®, e.g. Solvesso 200 (Exxon Mobil, USA), Aromatic, e.g. Aromatic 200 (Exxon Mobil), or Shellsol® products (Deutsche Shell Chemie GmbH, Germany). Particularly preferred as component (b3) are Solvesso 200 and Aromatic 200.

The aromatic hydrocarbon component (b3) generally amounts to 0 to 30% by weight, preferably 0 to 10% by weight, in particular 1 to 5% by weight of the formulation.

The preferred EC formulation also contains at least one emulsifier. The emulsifier serves to reduce surface tension between the continuous and the disperse phase, thereby stabilizing the droplets of the disperse phase. The emulsifier also assists in the solubilisation of the compound of formula (I). Suitable emulsifiers are well known in the art, e.g. from McCutcheon's Detergents and Emulsifiers, Int. Ed., Ridgewood, N.Y. Suitable emulsifiers include non-ionic, anionic, cationic and zwitterionic emulsifiers and mixtures thereof. The emulsifiers may be polymeric emulsifiers or non-polymeric emulsifiers. Non-polymeric emulsifiers, in contrast to polymeric emulsifiers, will generally have a molecular weight of below 2000 (number average), in particular from 150 to 2000, preferably from 200 to 1500.

The emulsifiers contained in the EC according to the invention can be nonionic or ionic, or a combination of both. It is preferred to use at least two, preferably three to five emulsifiers, preferably with different HLB values to achieve a good physicochemical behaviour of the EC at different temperatures.

The HLB (Hydrophile-Lipophile-Balance) is an empirical scale defined by W. C. Griffin (J. Soc. Cosmetic Chemists, 1, 311 (1949)) which expresses the amphiphilic nature of emulsifying agents (particularly nonionic emulsifiers). The least hydrophilic emulsifiers are assigned the lowest HLB values.

Suitable nonionic emulsifiers are, for example, alkoxylated fats or oils of animal or vegetable origin such as maize oil ethoxylates, castor oil ethoxylates, tallow fat ethoxylates, glycerol esters such as glycerol monostearate, fatty alcohol alkoxylates and oxoalcohol alkoxylates, fatty acid alkoxylates such as oleic acid ethoxylate, alkylphenyl alkoxylates such as isononyl-, isooctyl-, tributyl- and tristearylphenyl ethoxylates, fatty amine alkoxylates, fatty acid amide alkoxylates, sugar emulsifiers such as sorbitan fatty acid esters (sorbitan monooleate, sorbitan tristearate), polyoxyethylene sorbitan fatty acid esters, alkylpolyglycosides, N-alkylgluconamides, alkylmethyl sulfoxides, alkyldimethylphosphine oxides such as tetradecyldimethylphosphine oxide, ethylene oxide/propylene oxide copolymers and mixtures of such nonionic emulsifiers.

Preferred nonionic emulsifiers are, for example, sorbitan fatty acid esters, in particular partial esters of sorbitol and its anhydrides, e.g. sorbitan monooleate, polyoxyethylene sorbitan fatty acid esters, such as polyethoxylated (preferably with approximately 20 moles of ethylene oxide) sorbitan monolaurate and sorbitan monooleate, castor oil ethoxylates, preferably with approximately 40 moles of ethylene oxide), and ethylene oxide/propylene oxide copolymers, such as alkyl ethylene oxide/propylene oxide copolymers, preferably with a molecular weight in the range of 2000 to 5000.

Ionic emulsifiers can be anionic emulsifiers or cationic emulsifiers or mixtures of anionic and cationic emulsifiers.

Examples of anionic emulsifiers are phosphate esters and sulfate esters of poly (preferably 2 to 30) ethoxylated (preferably C₆ to C₂₂) fatty alcohols such as ethoxylated (2EO (EO means an ethylene oxyde unit) oleyl alcohol phosphate ester (e.g. Empiphos® O3D, Albright & Wilson, UK), ethoxylated oleyl alcohol phosphate esters (e.g. Crodafos® N serie, Croda Oleochemicals, UK), ethoxylated (2-10 EO) ceto/stearyl alcohol phosphate esters (e.g. Crodafos® CS serie, Croda Oleochemicals, UK), ethoxylated (4-6 EO) tridecyl alcohol phosphate esters (e.g. Emphos® PS serie, CK Witco, USA), ethoxylated fatty alcohol phosphate esters (e.g. Crafol® AP serie, Henkel lberica, Spain), ethoxylated (3-6 EO) fatty alcohol phosphate esters (e.g. Rhodafac® serie, Rhodia Chimie, France), free acids of complex organic phosphate esters (e.g. Beycostat® serie, Ceca S.A., France), phosphate esters of polyethoxylated (8 to 25 EO) arylphenols (such as polyethoxylated di- and tristyrylphenols) (e.g. Soprophor 3D33, Rhodia Chimie, France), sulfate esters of polyethoxylated arylphenols (such as polyethoxylated di- and tristyrylphenols) (e.g. Soprophor DSS/7, Soprophor 4D384, Rhodia Chimie, France).

Examples of cationic emulsifiers include alkyltrimethylammonium halides or alkyltrimethylammonium alkyl sulfates, alkylpyridinium halides or dialkyldimethylammonium halides and dialkyldimethylammonium alkyl sulfates.

Of the ionic emulsifiers anionic emulsifiers are preferred.

In a preferred embodiment of the invention, the emulsifier component comprises at least one emulsifier from the group of the sorbitan fatty monoesters, in particular sorbitan monooleate, and one or more, preferably two, emulsifiers from the group of the polyoxyethylene sorbitan fatty esters, in particular sorbitan monooleate and sorbitan monolaurate, each ethoxylated with approximately 20 moles ethylene oxide.

In a particularly preferred embodiment of the invention, the emulsifier component comprises an emulsifier from the group of the sorbitan fatty monoesters, one or more emulsifiers, preferably two, from the group of the polyethoxylated sorbitan fatty esters, and one or more emulsifiers from the group of the castor oil ethoxylates and ethylene oxide/propylene oxide copolymers.

The referenced nonionic emulsifiers are all commercially available. For example, sorbitan fatty acids are available as the S-MAZ® (BASF, Germany) or the Span® (UNIQEMA, US) series, polyoxyethylene sorbitan fatty esters as the T-MAZ® (BASF, Germany) or the Tween® (UNIQEMA, US) series, castor oil ethoxylates as Trylox 5909 (Cognis, Germany), and ethylene oxide/propylene oxide copolymers as the Tergitol series, such as Tergitol® XD (Dow, USA) or the Surfonic® LPP series.

The emulsifiers in the EC formulation generally amount to from 2 to 20% by weight, preferably 5 to 15% by weight of the formulation

In the preferred and particularly preferred embodiments, the sorbitan fatty monoesters generally amount to from 0.1 to 15% by weight, preferably 1 to 5% by weight of the formulation; the polyethoxylated sorbitan fatty esters generally amount to 1 to 5% by weight, preferably 1 to 5% by weight of the formulation, the polyethoxylated castor oil generally amounts to 0 to 15% by weight, preferably 0 to 5% by weight of the formulation, and the ethylene oxide/propylene oxide copolymer generally amounts to 0 to 15% by weight, preferably 0 to 5% by weight of the formulation.

In addition, the EC formulation according to the invention may comprise other conventional formulation additives, such as cosolvents, antifoams, antifreezes, preservatives, colorants, and wetting agents.

Suitable antifoams are, for example, aliphatic or aromatic monoalcohols having 4 to 14, preferably 6 to 10 carbon atoms, such as n-octanol or n-decanol, or silicone emulsifiers. The antifoams generally amount to from 0 to 10% by weight, preferably 0.01 to 1% by weight, of the formulation.

Typical antifreezes are, for example, ethylenglykol, propylenglykol, and glycerol.

Typical preservatives are, for example, vitamin E acetate, benzoic acid, sorbic acid, formaldehyde and traces of microbicidal compounds. Preservatives generally amount to from 0 to 10% by weight, preferably 0 to 1% by weight of the formulation.

Typical colorants include oil soluble dyes, such as Vitasyn® Patentblau (Clariant, Germany).

Typical wetting agents are, for example, polyethoxylated alkyl phenols (containing 1 to 30 moles ethylene oxide), polyethoxylated fatty alcohols (containing 1 to 30 moles ethylene oxide), tridecyl alcohol polyglykol ethers, and alkyl- or alkylphenyl-sulfonates. Wetting agents generally amount to from 0 to 50% by weight, preferably 0 to 10% by weight of the formulation.

The total content of further formulation additives generally amounts to from 0 to 52% by weight, preferably 0 to 10% by weight, more preferred 0 to 5% by weight of the formulation.

The EC formulation according to the invention is prepared in a manner known per se by mixing the components, if appropriate with stirring and/or heating. The products thus obtainable are normally homogeneous emulsion concentrates.

Containers which are suitable for the formulations are all containers conventionally used for crop protection products, mainly bottles, canisters, and bags made of chemical-resistant polymers. The use of water-soluble containers, mainly water-soluble film bags, in particular based on polyvinyl alcohol, is advantageous.

For application against pests the EC formulation is usually diluted with a suitable diluent, generally water, preferably with an at least 10 to 400, preferably 10 to 150 fold excess of diluent.

The mixture of the active compounds according to the invention can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the mixtures according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of mixtures, wetter, tackifier, dispersant or emulsifier and, if appropriate, solvent or oil, and such concentrates are suitable for dilution with water.

The concentrations of the mixtures of the active compounds in the ready-to-use preparations can be varied within relatively wide ranges. In general, they are from 0.0001 to 10%, preferably from 0.01 to 1%.

The mixtures of the active compounds may also be used successfully in the ultra-low-volume process (ULV), it being possible to apply formulations comprising over 95% by weight of active compound, or even to apply the mixtures of the active compound without additives.

As stated above, the mixture of this invention may also comprise other active ingredients, for example other pesticides, such as insecticides, fungicides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). These agents can be admixed with the mixtures according to the invention in a weight ratio of 1:10 to 10:1. For example, the plant(s) may be sprayed with a composition of this invention either before or after being treated with other active ingredients.

The mixtures and methods according to the invention are used for the control of pests, such as insects, acarids and nematodes. They can be applied to any and all developmental stages, such as egg, larva, pupa, and adult.

The pests may be controlled by contacting the pest itself, its food supply, habitat, breeding ground or its locus with a pesticidally effective amount of the inventive mixtures or of compositions comprising the mixtures.

“Locus” means a plant, seed, soil, area, material or environment in which a pest is growing or may grow.

In general, “pesticidally effective amount” means the amount of the inventive mixtures or of compositions comprising the mixtures needed to achieve an observable effect on growth, including the effects of necrosis, death, retardation, prevention, and removal, destruction, or otherwise diminishing the occurrence and activity of the target organism. The pesticidally effective amount can vary for the various mixtures/compositions used in the invention. A pesticidally effective amount of the mixtures/compositions will also vary according to the prevailing conditions such as desired pesticidal effect and duration, weather, target species, locus, mode of application, and the like.

The inventive mixtures or compositions of these mixtures can also be employed for protecting plants from attack or infestation by pests, such as insects, acarids or nematodes, comprising contacting a plant, or soil or water in which the plant is growing with a mixture or composition according to the invention in a pesticidally effective amount.

In the context of the present invention, the term plant refers to an entire plant, a part of the plant or the propagation material of the plant, such as the seed, the seed piece, the transplant, the seedling, or the cutting.

Plants which can be treated with the inventive mixtures include all genetically modified plants or transgenic plants, e.g. crops which tolerate the action of herbicides or fungicides or insecticides owing to breeding, including genetic engineering methods, or plants which have modified characteristics in comparison with existing plants, which can be generated for example by traditional breeding methods and/or the generation of mutants, or by recombinant procedures.

Some of the inventive mixtures have systemic action and can therefore be used for the protection of the plant shoot against foliar pests as well as for the treatment of the seed and roots against soil pests. The term seed treatment comprises all suitable seed treatment techniques known in the art, such as seed dressing, seed coating, seed dusting, seed soaking and seed pelleting.

The compounds (I) and (II) can be applied simultaneously, that is jointly or separately, or in succession, the sequence, in the case of separate application, generally not having any effect on the result of the control measures.

The compounds (I) and (II) are usually applied in a weight ratio of from 500:1 to 1:6000, preferably from 100:1 to 1:100, more preferably from 20:1 to 1:50, especially from 10:1 to 1:10, in particular from 5:1 to 1:20, very particularly between 5:1 to 1:5, particularly preferabyl between 2:1 and 1:2, also preferably between 4:1 and 2:1, mainly in the ratio of 1:1, or 5:1, or 5:2, or 5:3, or 5:4, or 4:1, or 4:2, or 4:3, or 3:1, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 3:75, or 4:75, or 1:6000, or 1:3000, or 1:1500, or 1:350, or 2:350, or 3:350, or 4:350, or 1:750, or 2:750, or 3:750, or 4:750.

Depending on the desired effect, the application rates of the mixtures according to the invention are from 5 g/ha to 2000 g/ha, preferably from 50 to 1500 g/ha, in particular from 50 to 750 g/ha.

The inventive mixtures are used for the protection of the seed, and the seedlings' roots and shoots, against soil pests.

Conventional seed treatment formulations include for example flowable concentrates FS, solutions LS, powders for dry treatment DS, water dispersible powders WS or granules for slurry treatment, water soluble powders SS and emulsion ES. Application to the seeds by contacting the seeds with a mixture or composition of the invention is carried out before sowing, either directly on the seeds or after having pregerminated the latter, at sowing or after sowing. Preferred are FS formulations.

In the treatment of seed, the application rates of the inventive mixture are generally from 0.1 g to 10 kg, preferably 1 g to 2 kg per 100 kg of seed. The separate or joint application of the compounds (I) and (II) or of the mixtures of the compounds (I) and (II) is carried out by spraying or dusting the seeds, the seedlings, the plants or the soils before or after sowing of the plants or before or after emergence of the plants.

The invention also relates to the propagation products of plants, and especially the seed comprising, that is, coated with and/or containing, a mixture as defined above or a composition containing the mixture of two or more active ingredients or a mixture of two or more compositions each providing one of the active ingredients. The seed comprises the inventive mixtures in an amount of from 0.1 g to 10 kg per 100 kg, preferably from 1 g to 5 kg per 100 kg, most preferably from 1 g to 2.5 kg per 100 kg, in particular 1 g to 2 kg of seed.

The inventive mixtures are effective through both contact (via soil, glass, wall, bed net, carpet, plant parts or animal parts), and ingestion (bait, or plant part) and through trophallaxis and transfer.

Preferred application methods are into water bodies, the soil, cracks and crevices, pastures, manure piles, sewers, into water, on floor, wall, or by perimeter spray application and bait.

According to a preferred embodiment of the invention, the inventive mixtures are employed via soil application. Soil application is especially favorable for use against ants, termites, flies, crickets, grubs, root weevils, root beetles or nematodes.

According to another preferred embodiment of the invention, for use against non crop pests such as ants, termites, wasps, flies, mosquitoes, crickets, locusts, or cockroaches the inventive mixtures are prepared into a bait preparation.

The bait can be a liquid, a solid or a semisolid preparation (e.g. a gel). The bait employed in the composition is a product which is sufficiently attractive to incite insects such as ants, termites, wasps, flies, mosquitoes, crickets etc. or cockroaches to eat it. This attractant may be chosen from feeding stimulants or para and/or sex pheromones. Suitable feeding stimulants are chosen, for example, from animal and/or plant proteins (meat-, fish- or blood meal, insect parts, crickets powder, egg yolk), from fats and oils of animal and/or plant origin, or mono-, oligo- or polyorganosaccharides, especially from sucrose, lactose, fructose, dextrose, glucose, starch, pectin or even molasses or honey, or from salts such as ammonium sulfate, ammonium carbonate or ammonium acetate. Fresh or decaying parts of fruits, crops, plants, animals, insects or specific parts thereof can also serve as a feeding stimulant. Pheromones are known to be more insect specific. Specific pheromones are described in the literature and are known to those skilled in the art.

Formulations of the inventive mixtures as aerosols (e.g in spray cans), oil sprays or pump sprays are highly suitable for the non-professional user for controlling pests such as flies, fleas, ticks, mosquitoes, locusts or cockroaches. Aerosol recipes are preferably composed of the active mixture, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The inventive mixtures and their respective compositions can also be used in mosquito coils and fumigating coils, smoke cartridges, vaporizer plates, long-term vaporizers, or other heat-independent vaporizer systems.

Methods to control infectious diseases transmitted by insects (e.g. malaria, dengue and yellow fever, lymphatic filariasis, and leishmaniasis) with the inventive mixtures and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The inventive mixtures and the compositions comprising them can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade houses and public facilities). The inventive mixtures are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant control composition of the present invention is directly applied to the nest of the ants or to its surrounding or via bait contact.

The compounds or compositions of the inventive mixtures can also be applied preventively to places at which occurrence of the pests is expected. In the case of soil treatment or of application to the pests dwelling place or nest, the quantity of the mixture of the active ingredients ranges from 0.0001 to 500 g per 100 m², preferably from 0.001 to 20 g per 100 m².

Customary application rates in the protection of materials are, for example, from 0.01 g to 1000 g of the mixture of the active compounds per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of the mixture of the active ingredients.

For use in bait compositions, the typical content of the mixture of active ingredients is from 0.0001 weight % to 15 weight %, desirably from 0.001 weight % to 5% weight % of active compounds. The composition used may also comprise other additives such as a solvent of the active materials, a flavoring agent, a preserving agent, a dye or a bitter agent. Its attractiveness may also be enhanced by a special color, shape or texture.

For use in spray compositions, the content of the mixture of the active ingredients is from 0.001 to 80 weights %, preferably from 0.01 to 50 weight % and most preferably from 0.01 to 15 weight %.

For use in treating crop plants, the rate of application of the mixture of the active ingredients of this invention may be in the range of 0.1 g to 4000 g per hectare, desirably from 25 g to 600 g per hectare, more desirably from 50 g to 500 g per hectare.

It was also an object of the present invention to provide mixtures suitable for treating, controlling, preventing and protecting warm-blooded animals, including humans, and fish against infestation and infection by pests. Problems that may be encountered with pest control on or in animals and/or humans are similar to those described at the outset, namely the need for reduced dosage rates, and/or enhanced spectrum of activity and/or combination of knock-down activity with prolonged control and/or resistance management.

This invention also provides a method for treating, controlling, preventing and protecting warm-blooded animals, including humans, and fish against infestation and infection by pests, preferably of the orders Siphonaptera, Hymenoptera, Hemiptera, Orthoptera, Acarina, Phthiraptera, and Diptera, which comprises orally, topically or parenterally administering or applying to said animals a pesticidally effective amount of mixtures or compositions according to the invention.

The invention also provides a process for the preparation of a composition for controlling pests and for treating, preventing or protecting a warm-blooded animal or a fish against infestation or infection by pests, said pests being preferably of the Siphonaptera, Hymenoptera, Hemiptera, Orthoptera, Acarina, Phthiraptera, and Diptera orders, which comprises mixing a pesticidally effective amount of compounds (I) and (II) and optionally custuomary formulation aids.

The above method is particularly useful for controlling and preventing infestations and infections in warm-blooded animals such as cattle, sheep, swine, camels, deer, horses, poultry, goats, dogs and cats as well as humans.

Further provided is the use of a pesticidally effective amount of the compounds (I) and (II) and optionally further formulation aids for preparing the above composition.

Infestations in warm-blooded animals and fish including, but not limited to, lice, biting lice, ticks, nasal bots, keds, biting flies, muscoid flies, flies, myiasitic fly larvae, chiggers, gnats, mosquitoes and fleas may be controlled, prevented or eliminated by the mixtures according to the invention.

For oral administration to warm-blooded animals, the mixtures according to the invention may be formulated as animal feeds, animal feed premixes, animal feed concentrates, pills, solutions, pastes, suspensions, drenches, gels, tablets, boluses and capsules. In addition, the mixtures according to the invention may be administered to the animals in their drinking water. For oral administration, the dosage form chosen should provide the animal with 0.01 mg/kg to 100 mg/kg of animal body weight per day of the mixture.

Alternatively, the mixtures according to the invention may be administered to animals parenterally, for example, by intraruminal, intramuscular, intravenous or subcutaneous injection. The mixtures according to the invention may be dispersed or dissolved in a physiologically acceptable carrier for subcutaneous injection. Alternatively, the mixtures according to the invention may be formulated into an implant for subcutaneous administration. In addition the mixtures according to the invention may be transdermally administered to animals. For parenteral administration, the dosage form chosen should provide the animal with 0.01 mg/kg to 100 mg/kg of animal body weight per day of the mixture.

The mixtures according to the invention may also be applied topically to the animals in the form of dips, dusts, powders, collars, medallions, sprays, spot-on and pour-on formulations. For topical application, dips and sprays usually contain 0.5 ppm to 5,000 ppm and preferably 1 ppm to 3,000 ppm of the inventive compounds. In addition, the mixtures according to the invention may be formulated as ear tags for animals, particularly quadrupeds such as cattle and sheep.

Accordingly, in a further aspect of the invention there is provided the use of a mixture according to the invention in the preparation of a veterinary medicament, specifically an antiparasiticidal medicament.

The pesticidal action of the mixtures according to the invention can be demonstrated by one or more of the experiments below:

Bean Aphid (aphis fabae)

The active compounds are formulated in 50:50 acetone:water and 100 ppm Kinetic® surfactant.

Nasturtium plants grown in Metro mix in the 1^{st} leaf-pair stage (variety ‘Mixed Jewel’) are infested with approximately 2-30 laboratory-reared aphids by placing infested cut plants on top of the test plants. The cut plants are removed after 24 hr. Each plant is dipped into the test solution to provide complete coverage of the foliage, stem, protruding seed surface and surrounding cube surface and allowed to dry in the fume hood. The treated plants are kept at about 25° C. with continuous fluorescent light. Aphid mortality is determined after 3 days.

Boll Weevil (Anthonomus grandis)

The active compounds are formulated in 1:3 DMSO:water. 10 to 15 eggs are placed into microtiter plates filled with 2% agar-agar in water and 300 ppm formaline. The eggs are sprayed with 20 μl of the test solution, the plates are sealed with pierced foils and kept at 24-26° C. and 75-85% humidity with a day/night cycle for 3 to 5 days. Mortality is assessed on the basis of the remaining unhatched eggs or larvae on the agar surface and/or quantity and depth of the digging channels caused by the hatched larvae. Tests are replicated 2 times.

Brown Planthopper (nilaparvata lugens)

The active compounds are formulated in 50:50 acetone:water. Potted rice seedlings are sprayed with 10 ml test solution, air dried, placed in cages and inoculated with 10 adults. Percent mortality is recorded after 24, 72 and 120 hours.

Colorado Potato Beetle (Leptinotarsa decemlineata)

Potato plants are utilized for bioassays. Excised plant leaves are dipped into 1:1 acetone/water dilutions of the active compounds. After the leaves have dried, they are individually placed onto water-moistened filter paper on the bottoms of Petri dishes. Each dish is infested with 5-7 larvae and covered with a lid. Each treatment dilution is replicated 4 times. Test dishes are held at approximately 27° C. and 60% humidity. Numbers of live and morbid larvae are assessed in each dish at 5 days after treatment application, and percent mortality is calculated.

Cotton Aphid (aphis gossypii)

The active compounds are formulated in 50:50 acetone:water and 100 ppm Kinetic® surfactant.

Cotton plants at the cotyledon stage (one plant per pot) are infested by placing a heavily infested leaf from the main colony on top of each cotyledon. The aphids are allowed to transfer to the host plant overnight, and the leaf used to transfer the aphids is removed. The cotyledons are dipped in the test solution and allowed to dry. After 5 days, mortality counts are made.

Cowpea Aphid (aphis craccivora)

The active compounds are formulated in 50:50 acetone:water. Potted cowpea plants colonized with 100-150 aphids of various stages are sprayed after the pest population has been recorded. Population reduction is recorded after 24, 72, and 120 hours.

Diamond Back Moth (plutella xylostella)

The active compounds are formulated in 50:50 acetone:water and 0.1% (vol/vol) Alkamuls EL 620 surfactant. A 6 cm leaf disk of cabbage leaves is dipped in the test solution for 3 seconds and allowed to air dry in a Petri plate lined with moist filter paper. The leaf disk is inoculated with 10 third instar larvae and kept at 25-27° C. and 50-60% humidity for 3 days. Mortality is assessed after 72 h of treatment.

Green Peach Aphid (Myzus persicae

The active compounds are formulated in 50:50 acetone:water and 100 ppm Kinetic® surfactant.

Pepper plants in the 2^{nd} leaf-pair stage (variety ‘California Wonder’) are infested with approximately 40 laboratory-reared aphids by placing infested leaf sections on top of the test plants. The leaf sections are removed after 24 hr. The leaves of the intact plants are dipped into gradient solutions of the test compound and allowed to dry. Test plants are maintained under fluorescent light (24 hour photoperiod) at about 25° C. and 20-40% relative humidity. Aphid mortality on the treated plants, relative to mortality on check plants, is determined after 5 days.

Mediterranean Fruitfly (Ceratitis capitata)

The active compounds are formulated in 1:3 DMSO:water. 50 to 80 eggs are placed into microtiter plates filled with 0.5% agar-agar and 14% diet in water. The eggs are sprayed with 5 μl of the test solution, the plates are sealed with pierced foils and kept at 27-29° C. and 75-85% humidity under fluorescent light for 6 days. Mortality is assessed on the basis of the agility of the hatched larvae. Tests are replicated 2 times.

Rice Green Leafhopper (Nephotettix virescens)

Rice seedlings are cleaned and ished 24 hours before spraying. The active compounds are formulated in 50:50 acetone:water, and 0.1% vol/vol surfactant (EL 620) is added. Potted rice seedlings are sprayed with 5 ml test solution, air dried, placed in cages and inoculated with 10 adults. Treated rice plants are kept at 28-29° C. and relative humidity of 50-60%. Percent mortality is recorded after 72 hours.

Rice Plant Hopper (Nilaparvata lugens)

Rice seedlings are cleaned and ished 24 hours before spraying. The active compounds are formulated in 50:50 acetone:water and 0.1% vol/vol surfactant (EL 620) is added. Potted rice seedlings are sprayed with 5 ml test solution, air dried, placed in cages and inoculated with 10 adults. Treated rice plants are kept at 28-29° C. and relative humidity of 50-60%. Percent mortality is recorded after 72 hours.

Silverleaf Whitefly (bemisia argentifolii)

The active compounds are formulated in 50:50 acetone:water and 100 ppm Kinetic® surfactant.

Selected cotton plants are grown to the cotyledon state (one plant per pot). The cotyledons are dipped into the test solution to provide complete coverage of the foliage and placed in a well-vented area to dry. Each pot with treated seedling is placed in a plastic cup and 10 to 12 whitefly adults (approximately 3-5 day old) are introduced. The insects are colleted using an aspirator and an 0.6 cm, non-toxic TygonÒ tubing (R-3603) connected to a barrier pipette tip. The tip, containing the collected insects, is then gently inserted into the soil containing the treated plant, allowing insects to crawl out of the tip to reach the foliage for feeding. The cups are covered with a re-usable screened lid (150 micron mesh polyester screen PeCap from Tetko Inc). Test plants are maintained in the holding room at about 25° C. and 20-40% relative humidity for 3 days avoiding direct exposure to the fluorescent light (24 hour photoperiod) to prevent trapping of heat inside the cup. Mortality is assessed 3 days after treatment of the plants.

Southern Armyworm (Spodoptera eridania), 2^{nd} Instar Larvae

The active compounds are formulated for testing the activity against insects and arachnids as a 10.000 ppm solution in a mixture of 35% acetone and water, which is diluted with water, if needed.

A Sieva lima bean leaf expanded to 7-8 cm in length is dipped in the test solution with agitation for 3 seconds and allowed to dry in a hood. The leaf is then placed in a 100×10 mm petri dish containing a damp filter paper on the bottom and ten 2^{nd} instar caterpillars. At 5 days, observations are made of mortality, reduced feeding, or any interference with normal molting.

Tobacco Budworm (Heliothis virescens)

Two-leaf cotton plants are utilized for bioassays. Excised plant leaves are dipped into 1:1 acetone/water dilutions of the active compounds. After the leaves have dried, they are individually placed onto water-moistened filter paper on the bottoms of Petri dishes. Each dish is infested with 5-7 larvae and covered with a lid. Each treatment dilution is replicated 4 times. Test dishes are held at approximately 27° C. and 60% humidity. Numbers of live and morbid larvae are assessed in each dish at 5 days after treatment application, and percent mortality is calculated.

2-Spotted Spider Mite (Tetranychus urticae, OP-Resistant Strain) The active compounds are formulated in 50:50 acetone:water and 100 ppm Kinetic® surfactant.

Sieva lima bean plants with primary leaves expanded to 7-12 cm are infested by placing on each a small piece from an infested leaf (with about 100 mites) taken from the main colony. This is done at about 2 hours before treatment to allow the mites to move over to the test plant to lay eggs. The piece of leaf used to transfer the mites is removed. The newly-infested plants are dipped in the test solution and allowed to dry. The test plants are kept under fluorescent light (24 hour photoperiod) at about 25° C. and 20-40% relative humidity. After 5 days, one leaf is removed and mortality counts are made.

Vetch Aphid (Megoura viciae)

The active compounds are formulated in 1:3 DMSO:water. Bean leaf disks are placed into microtiter plates filled with 0.8% agar-agar and 2.5 ppm OPUS™. The leaf disks are sprayed with 2.5 μl of the test solution and 5 to 8 adult aphids are placed into the microtiterplates which are then closed and kept at 22-24° C. and 35-45% under fluorescent light for 6 days. Mortality is assessed on the basis of vital, reproduced aphids. Tests are replicated 2 times.

Wheat Aphid (Rhopalosiphum padi)

The active compounds are formulated in 1:3 DMSO:water. Barlay leaf disk are placed into microtiterplates filled with 0.8% agar-agar and 2.5 ppm OPUS™. The leaf disks are sprayed with 2.5 μl of the test solution and 3 to 8 adult aphids are placed into the microtiterplates which are then closed and kept at 22-24° C. and 35-45% humidity under fluorescent light for 5 days. Mortality is assessed on the basis of vital aphids. Tests are replicated 2 times.

### Nematicidal Evaluation

Test compounds are prepared and formulated into aqueous formulations using 5% acetone and 0.05% TWEEN 20 (polyoxyethylene (2) sorbitan monolaureate) as a surfactant.

Test Procedures for root-knot nematode (Meloidogyne hapla and Meloidogyne incognita):

Tomato (variety Bonny Best) seeds are germinated in flats, then at the first true-leaf stage seedlings are transferred to planting cells. The soil in the cells is a 1:1 mix of sandy loam and coarse sand. The transplants are maintained in the greenhouse for one week. Compounds are applied as a soil drench, 1 ml per planting cell. Each treatment is replicated three times. Later the same day, plants are inoculated with an aqueous suspension of J2 nematodes consisting of a mixed population of two root-knot nematodes, Meloidogyne hapla and M. incognita, 1 ml with 1000 J2s per cell. Plants are kept in a moist infection chamber for 1 day following inoculation, then moved to a greenhouse and bottom-watered until the root systems are harvested for evaluation.

Two weeks after inoculation, tomato root systems are harvested and the number of root-knot galls on each root system are counted.

Nematicidal activity is calculated as the percent reduction in root-knot galls as follows where:
T=The median number of root-knot galls for a treatment.
SB=The median number of root-knot galls for the solvent blank control.

Percent reduction in root-knot galling=((SB−T)/SB)*100%

Eastern Subterranean Termites (Reticulitérmes flávipes) and Formosan subterranean Termites (Coptotermes formosanus)

Toxicant treatments (1.0% test compound w/w) are applied to 4.25 cm (diam.) filter papers (VWR #413, qualitative) in acetone solution. Treatment levels (% test compound) are calculated on basis of a mean weight per filter paper of 106.5 mg. Treatment solutions are adjusted to provide the quantity of toxicant (mg) required per paper in 213 ml of acetone (volume required for saturation of paper). Acetone only is applied for untreated controls. Treated papers are vented to evaporate the acetone, moistened with 0.25 ml water, and enclosed in 50×9 mm Petri dishes with tight-fit lids (3-mm hole in side of each dish for termite entry).

Termite bioassays are conducted in 100×15 mm Petri dishes with 10 g fine sand spread in a thin layer over the bottom of each dish. An additional 2.5 g sand is piled against the side of each dish. The sand is moistened with 2.8 ml water applied to the piled sand. Water is added to dishes as needed over the course of the bioassays to maintain high moisture content. Bioassays are done with one treated filter (inside enclosure) and 30 termite workers per test dish. Each treatment level is replicated in 2 test dishes. Test dishes are maintained at about 25° C. and 85% humidity for 12 days and observed daily for mortality.

Orchid Thrips (Dichromothrips corbetti)

Dichromothrips corbetti adults used for bioassay are obtained from a colony maintained continuously under laboratory conditions. For testing purposes, the test compound is diluted to a concentration of 500 ppm (wt compound: vol diluent) in a 1:1 mixture of acetone:water, plus 0.01% Kinetic surfactant.

Thrips potency of each compound is evaluated by using a floral-immersion technique. Plastic petri dishes are used as test arenas. All petals of individual, intact orchid flowers are dipped into treatment solution for approximately 3 seconds and allowed to dry for 2 hours. Treated flowers are placed into individual petri dishes along with 10-15 adult thrips. The petri dishes are then covered with lids. All test arenas are held under continuous light and a temperature of about 28° C. for duration of the assay. After 4 days, the numbers of live thrips are counted on each flower, and along inner walls of each petri dish. The level of thrips mortality is extrapolated from pre-treatment thrips numbers.

Yellow Fever Mosquitos (Aedes aegypti)

The test compound (1 Vol % in acetone) is applied to water in glass dishes containing 4^{th}-instar Aedes aegypti. The test dishes are maintained at about 25° C. and observed daily for mortality. Each test is replicated in 3 test dishes.

### Test Methodology

1. Activity against argentine ant, harvester ant, acrobat ant, carpenter ant, fire ant, House Fly, Stable Fly, Flesh Fly, Yellow fever Mosquito, House Mosquito, Malaria Mosquito, German Cockroach, Cat Flea, and Brown Dog Tick Via Glass Contact

Glass vials (20 ml scintillation vials) are treated with 0.5 ml of a solution of active ingredient in acetone. Each vial is rolled uncapped for ca. 10 minutes to allow the a.i. to completely coat the vial and to allow for full drying of the acetone. Insects or ticks are placed into each vial. The vials are kept at 22° C. and are observed for treatment effects at various time intervals.

### 2. Activity Against Argentine Ant, Acrobat Ant, Carpenter Ant, Fire Ant, And Eastern Subterranean Termite Via Soil Contact

For ants, tests are conducted in Petri dishes. A thin layer of 1% agar in water is dispensed into the dishes and Florida sandy soil is spread over the agar (5 g for the small dishes and 11 g for the larger dishes). The active ingredient is dissolved in acetone and dispensed over the sand. Dishes are vented to evaporate the acetone, infested with ants, and covered. A 20% honey water solution is placed in each dish. The dishes are maintained at 22° C. and observed for mortality at various time intervals.

For termites, a thin layer of 1% agar is dispensed into Petri dishes. A thin layer of pre-treated soil is spread over the agar. For soil treatment, the active ingredient is diluted in acetone on a weight-to-weight basis and incorporated into 100 g of soil. The soil is placed in a jar and vented for 48 hours. The moisture level of the soil is brought to field capacity by adding 7 ml of water. Termite workers are introduced into each dish. A small piece of filter paper is placed into each dish after 1 day as a food source, and additional water is added as needed to maintain soil moisture. Test dishes are held at a dark incubator at 25° C. and appr. 80% relative humidity. Termites are observed daily for mortality (dead or unable to stand upright and showing only weak movement).

### 3. Activity Against Argentine Ant, Acrobat Ant, Carpenter Ant, Fire Ant, House Fly, Eastern Subterranean Termite, Formosan Subterranean Termite, and German Cock-Roach Via Bait

For Argentine ant, acrobat ant, and carpenter ant, tests are conducted in Petri dishes. Ants are given a water source, and then are starved of a food source for 24 hours. Baits are prepared with either 20% honey/water solutions or ground cat chow. Active ingredient in acetone is added to the bait. 0.2 ml of treated honey water solution or 150 mg of treated cat chow, placed in a cap, is added to each dish. The dishes are covered and maintained at a temperature of 22° C. The ants are observed for mortality daily.

For the fire ants, corn grit is used as a bait matrix. Corn grit bait is prepared using a mixture of defatted corn grit (80%), soybean oil (19.9%), acetone, and the active ingredient (0.1%). Petri dishes are supplied with a water source. Fire ant adults are placed into each dish. The next day, 250 mg of bait in bait containers is placed into the dishes. The ants are observed for mortality daily.

For house flies. Bait tests are conducted with adults aged 2-5 days post-emergence. Active ingredient in acetone is applied to a bait matrix consisting of a 1:1 mixture of powdered milk and sugar which is then allowed to dry. Assays are conducted in jars with 250 mg of bait in a pan placed in the bottom of each jar. House flies are placed into the bait jars which are covered. The test jars are held at 22° C. Test jars are observed at 4 hours after treatment for knockdown (death plus morbidity (unable to stay upright).

For termites, active ingredient in acetone is applied to filter papers. % a.i. are calculated on basis of the weight of the filter paper. Acetone only is applied for untreated controls. Treated papers are vented to evaporate the acetone, moistened with ml water, and placed Petri dishes with sand. Water is added during the test as needed. Bioassays are conducted with one treated filter and ca. 30 termite workers per test dish. Test dishes are maintained at 25° C. and appr. 85% relative humidity and observed daily for mortality (dead or moribund insects) or intoxication. Dead or moribund insects are removed daily.

For cockroaches, plastic roach boxes with ventilated lids are used as test arenas. The top 3-4 cm of the arenas is treated with Vaseline and mineral oil to prevent roaches from escaping. Water is provided as needed. The bait is prepared using ground cat chow, and the active ingredient in acetone is incorporated on a weight-to-weight ratio. The treated chow is allowed to dry. The cockroaches are placed in the boxes and starved for 24 hours prior to bait introduction. 0.03 grams of bait per box are placed in a weigh boat. The boxes are maintained at 22° C. and observed daily for mortality of the cockroaches.

4. Activity Against Yellow fever Mosquito, Southern House Mosquito, and Malaria Mosquito Larvae Via Water Treatment

Well plates are used as test arenas. The active ingredient is dissolved in acetone and diluted with water to obtain the concentrations needed. The final solutions containing appr. 1% acetone are placed into each well. Approximately 10 mosquito larvae (4^{th}-instars) in 1 ml water are added to each well. Larvae are fed one drop of liver powder each day. The dishes are covered and maintained at 22° C. Mortality is recorded daily and dead larvae and live or dead pupae are removed daily. At the end of the test remaining live larvae are recorded and percent mortality is calculated.

Each test is replicated at least 3 times.

To determine if a pesticidal mixture is synergistic, Limpel's formula is used:

E=X+Y−XY/100

E=Expected % mortality of the mixture

X=% mortality of compound X, as measured independently

Y=% mortality of compound Y, as measured independently

Synergism is evident if the % observed mortality for the mixture is greater than the % expected mortality.

Test results show that the mixtures according to the invention show a considerable enhanced activity demonstrating synergism compared to the calculated sum of the single activities.

1-12. (canceled)

13: A pesticidal composition comprising, as active components,
A) a phenylsemicarbazone compound of the formula (I), 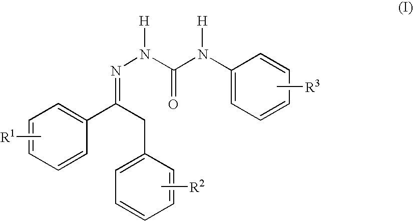
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 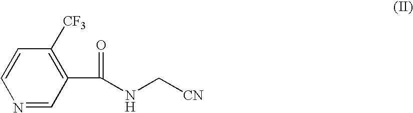
or an agriculturally acceptable salt thereof,
and
C) a liquid or solid carrier.

14: The pesticidal composition of claim 13, wherein the compound of formula (I) is metaflumizone.

15: The pesticidal composition of claim 13, comprising the compound of the formula (I) and the compound of the formula (II) in a weight ratio of from 100:1 to 1:100.

16: A pesticidal emulsifiable concentrate formulation comprising
I) a pesticidal composition comprising
A) a phenylsemicarbazone compound of the formula (I), 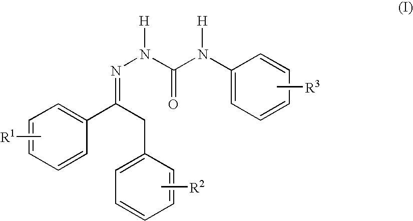
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 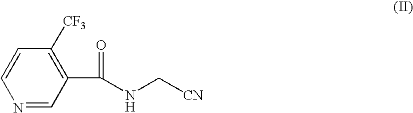
or an agriculturally acceptable salt thereof,
and
C) a liquid or solid carrier;
II) a solvent system, comprising
b1) γ-butyrolactone,
b2) one or more aliphatic and/or aromatic ketone, and
b3) optionally one or more aromatic hydrocarbon;
III) one or more emulsifier; and,
IV) optionally, further formulation additives.

17: A method for controlling pests comprising contacting the pests or their food supply, habitat, breeding grounds or their locus with a pesticidally effective amount of a composition comprising:
A) a phenylsemicarbazone compound of the formula (I), 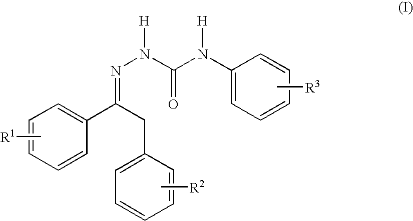
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 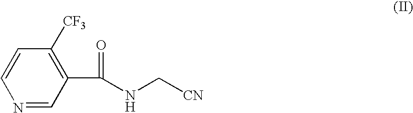
or an agriculturally acceptable salt thereof,
and
C) a liquid or solid carrier.

18: A method for protecting plants from attack or infestation by pests comprising contacting a plant, or soil or water in which the plant is growing, with a pesticidally effective amount of a composition comprising:
A) a phenylsemicarbazone compound of the formula (I), 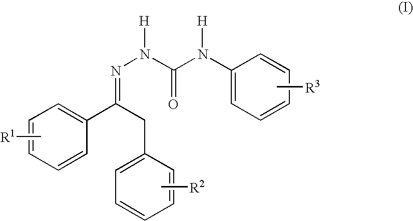
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 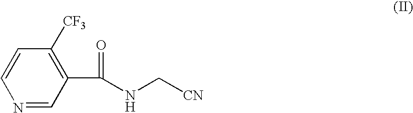
or an agriculturally acceptable salt thereof,
and
C) a liquid or solid carrier.

19: The method of claim 18, wherein the composition is applied in an amount of from 5 g/ha to 2000 g/ha.

20: A method of protection of seed comprising contacting the seeds before sowing and/or after peregrination with a pesticidally effective amount of a composition comprising:
A) a phenylsemicarbazone compound of the formula (I), 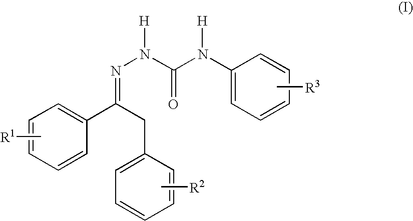
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 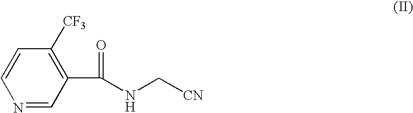
or an agriculturally acceptable salt thereof,
and
C) a liquid or solid carrier.

21: Seeds comprising in an amount of 0.1 g to 10 kg per 100 kg of seeds, a composition comprising:
A) a phenylsemicarbazone compound of the formula (I), 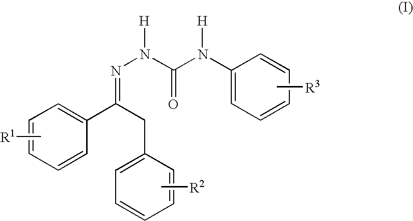
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 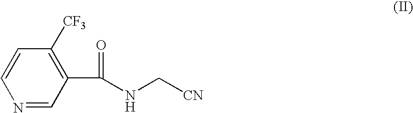
or an agriculturally acceptable salt thereof,
and
C) a liquid or solid carrier.

22: A method for treating, controlling, preventing or protecting a warm-blooded animal or a fish against infestation or infection by pests which comprises orally, topically or parenterally administering or applying to said animal or fish a pesticidally effective amount of a composition comprising:
A) a phenylsemicarbazone compound of the formula (I), 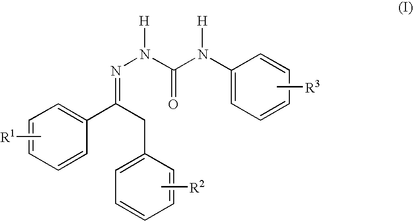
where R¹ and R² are, independently of one another, hydrogen, cyano, halogen, C₁-C₄-alkyl, C₁-C₄-alkoxy, C₁-C₄-haloalkyl or C₁-C₄-haloalkoxy and R³ is C₁-C₄-alkoxy, C₁-C₄-haloalkyl or C₁-C₄-haloalkoxy,
or an agriculturally acceptable salt thereof, and
B) a compound of the formula (II), 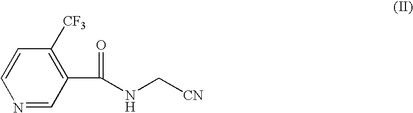
or an agriculturally acceptable salt thereof,
and
C) a liquid or solid carrier.
